(19) United States
(12) Patent Application Publication
(10) Pub. No.: US 2024/0038401 A1
(43) Pub. Date: Feb. 1, 2024

(54) METHOD FOR PREDICTING RESPONSE OF A SUBJECT TO ANTIDEPRESSANT TREATMENT

(71) Applicant: TALIAZ LTD., Tel Aviv (IL)

(72) Inventor: Dekel TALIAZ, Modi'in (IL)

(21) Appl. No.: 18/022,459

(22) PCT Filed: Aug. 19, 2021
(86) PCT No.: PCT/IL2021/051018

§ 371 Date: Feb. 21, 2023

Related U.S. Application Data
(60) Provisional application No. 63/070,006, filed on Aug. 25, 2020.

(51) Int. Cl. G16H 50/70; A61K 31/137; A61K 31/135; A61K 31/343

(52) U.S. Cl. CPC G16H 50/70; A61K 31/137; A61K 31/135; A61K 31/343

(57) ABSTRACT

Methods for predicting antidepressant treatment response for a subject in need thereof, for predicting response to antidepressant treatment, and for generating a predictor of response to antidepressant treatment, are provided.

### FIELD OF THE INVENTION

The invention is directed to systems and methods for predicting response to antidepressant treatment in a subject in need thereof.

### BACKGROUND OF THE INVENTION

Mood disorders are among the most prevalent forms of mental illness. Severe forms of mental illness affect 2%-5% of the US population and up to 20% of the population suffers from milder forms of the illness (Nestler et al., 2002, Neuron 34, 13-5). The economic costs to society and personal costs to individuals and families are enormous.

Anti-depressants are a primary method for treatment of depression. Anti-depressant drugs are known to influence the functioning of certain monoamine neurotransmitters, primarily serotonin, norepinephrine, and dopamine. Older medications, such as tricyclic anti-depressants (TCAs) and monoamine oxidase inhibitors (MAOIs), affect the activity of all these neurotransmitters simultaneously. However, these medications can be difficult to tolerate due to side effects or, in the case of MAOIs, dietary and medication restrictions. Newer medications, such as selective serotonin reuptake inhibitors (SSRIs) norepinephrine reuptake inhibitors (NRIs), Serotonin-norepinephrine reuptake inhibitors (SNRIs), Norepinephrine-Dopamine Reuptake Inhibitors (NDRIs) and Serotonin-Norepinephrine-Dopamine Reuptake Inhibitors (SNDRIs), Mirtazapine, Nefazodone, Trazodone, and Vortioxetine also have side-effects, though fewer. Prescription of anti-depressant medication is often inexact and their efficacy is assessed empirically. Depression, as well as other prevalent psychiatric disorders, is characterized by a high degree of variability in patient response to the drugs administered, even among individuals with the same diagnosis. In fact, only roughly 35% of patients demonstrate complete remission following first prescribed treatment. Furthermore, some patients respond, but with serious adverse side effects (Nestler et al., 2002, Neuron 34, 13-25).

Current methods for selecting a suitable depression treatment are basically trial-and-error. Patients will often have to be treated with several kinds of medicine, before finding the most suitable drug. This is a problem in itself, which is further augmented by the fact that four to six weeks of chronic treatment are required to evaluate the anti-depressant phenotype, the efficacy of the treatment and whether an adverse event is registered. It is therefore not surprising that patients tend to cease taking their medications against medical advice.

Forty to fifty percent of the risk for depression is genetic, making depression a highly heritable disorder. However, the search for a single gene responsible for major depressive disorder has given way to the understanding that depression is a complex disease in which multiple gene variants, each having only a slight contribution to the disorder, are involved (Nestler et al., 2002, Neuron 34, 13-25). The explanation to the variation in treatment efficacy of the different anti-depressants is also most probably due to the different genetic background of the patients. However, the search for gene variants explaining this variance has been of limited success.

However, there remains an unmet medical need for a method capable of predicting the efficacy and adverse effects of an anti-depressant treatment in a patient suffering from a psychiatric disorder such as depression, taking into account clinical as well as demographic information. This is important in order to shorten the time required to achieve an optimal treatment regime with minimal adverse side effects.

### SUMMARY OF THE INVENTION

The invention is directed to methods for predicting efficacy and adverse effects of an anti-depressant treatment in a patient suffering from a psychiatric disorder, such as depression. The invention is also directed to methods for predicting treatment resistance to anti-depressants of a subject suffering from a psychiatric disorder.

Advantageously, the methods disclosed herein enable predicting the patient's response to treatment with antidepressants prior to the patient being treated with antidepressants. That is, the methods enable predicting the patient's treatment response based on a combination of clinical and/or demographic data, wherein the clinical data may be obtained, for example, through a patient' answers to a questionnaire. In particular embodiments, the methods of the invention are exemplified herein for prediction of treatment resistance, and for the prediction of efficacy and side effects to antidepressant drugs, such as but not limited to citalopram (CELEXA™, CIPRAMIL®), bupropion, sertraline, venlafaxine (EFFEXOR™) and escitalopram.

The present invention provides, in one aspect, a method for predicting antidepressant treatment response of a subject in need thereof, the method comprising obtaining at least one of a clinical feature of the subject and/or a demographic feature of the subject, and processing the at least one clinical feature and/or demographic feature by applying a classification algorithm, the classification algorithm configured to provide a graduated score indicative of the treatment response to an antidepressant treatment.

According to some embodiments, at least one clinical feature is selected from the group consisting of: presence and/or severity level of any one of problems in the upper gastro intestine, pains or aches at different body parts, neurological issues, reported fear of having anxiety attack, history of psychotropic medications, poor treatment response to other antidepressants, reported troubling thoughts, reported sleep disorder, reported traumatic thoughts and effects, reported fear of public, open, and/or overpopulated spaces (Agoraphobia), and any combination thereof. Each possibility is a separate embodiment.

According to some embodiments, at least one demographic feature is selected from the group consisting of: employment status, residence, private health care insurance, age, marital status, and any combination thereof In some embodiments, the demographic feature relates to employment status, residence and age, or the demographic feature relates to employment status and having private healthcare insurance. Each possibility is a separate embodiment.

In some embodiments, any one of the clinical features and/or the demographic features can be divided into sub-features. In some embodiments, obtaining at least one of a clinical feature and/or a demographic feature of the subject comprises obtaining at least one sub-feature of a clinical feature and/or at least one sub-feature of a demographic feature of the subject. In some embodiments, each feature can be divided into two or more sub-features. In some embodiments, the method comprises obtaining a combination of sub-features associated with one or more of a clinical feature and/or a demographic feature. In some embodiments, the method comprises creating a new feature using a combination of features associated with one or more of a clinical feature and/or a demographic feature.

In some embodiments, the sub-features comprise different levels of severity of a clinical feature of a subject. For example, the severity of one or more clinical features can be divided into a scale, such as a scale ranging from 0 to 10, thereby providing 11 sub-features associated with the severity of a clinical feature. In some embodiments, the sub-features comprise different locations associated with a clinical feature of a subject. For example, in some embodiments, a clinical feature of a subject is divided into a plurality of sub-features wherein each feature is associated with a different body part of the subject.

In some embodiments, a demographic feature of a subject is divided into sub-features associated with specific details of the demographic feature. In some embodiments, the sub-features comprise different statuses associated with demographic features of a subject. In some embodiments, a demographic feature of a subject is divided into sub-features associated with a range of complexity associate with a demographic feature. For example, a marital status can be divided into sub-features relating to number of children in custody of the subject.

In some embodiments, the sub-features are associated with a time and/or duration of the demographic feature. In some embodiments, the sub-features are associated with a history of a subject regarding the demographic feature. For example, in some embodiments, the demographic features such as employment status and/or having private healthcare insurance, are divided into sub-features associated with the time at which each of the demographic features had changed, and the status of the change itself.

In some embodiments, the at least one clinical feature comprises a plurality of clinical features, selected from: severity level of problems in the upper gastro intestine, reported pains or aches in different body parts, presence and/or severity level of problems in the musculoskeletal/integument system, severity level of problems in the neurological system, fear of having an anxiety attack, reported feeling of unease, reported fear of having an anxiety attack, avoiding doing something because of fear from having an anxiety attack, fear of having an anxiety attack when traveling in a bus, train, or plane, being jumpy and easily startled because of having experienced a traumatic event, having a history of psychotropic medications, having a poor treatment response to other antidepressants, reported sleep disorder, reported traumatic thoughts and effects, reported fear of public, open, and/or overpopulated spaces (Agoraphobia), and reported troubling thoughts.

According to some embodiments, the method does not require obtaining any genetic information. According to some embodiments, only clinical features and/or demographic features as defined herein are obtained in the method. According to some embodiments, solely (no more than) clinical and/or demographic features are obtained. Advantageously, this enables providing a quick assessments of the patient's treatment response without having to await for the patient's genetic data and/or analysis of same.

According to some embodiments, the method comprises obtaining at least three clinical and/or demographic features. In some embodiments, the method comprises obtaining at least five clinical and/or demographic features. In some embodiments, the method comprises obtaining at least ten clinical and/or demographic features.

According to some embodiments, the method further comprises determining side effects of the antidepressant treatment.

According to some embodiments, the method comprises applying the at least one clinical feature and/or demographic feature to a machine learning algorithm. According to some embodiments, applying the at least one clinical feature and/or demographic feature to a machine learning algorithm comprises applying the at least one clinical feature and/or demographic feature to an ensemble predictor. According to some embodiments, the ensemble predictor is derived from applying the machine learning algorithm on a data set of the at least one clinical feature and/or demographic feature obtained from patients with a known treatment response, thereby obtaining score indicative of the subject's treatment response. In some embodiments, applying machine learning algorithm comprises a step of feature selection. According to some embodiments, the machine learning algorithm is configured to select features.

According to some embodiments, the classification algorithm comprises a non-linear classification algorithm. In some embodiments, the non-linear classification algorithm comprises an ensemble of classification and regression trees, more preferably wherein said ensemble of classification and regression trees, comprises a random forest classifier, support vector machine (SMV), partitioning around medoids (PAM) or a boosting framework; or wherein the graduated score has an accuracy of above 0.5 and a p-value for the accuracy of below 0.05 and an AUC of above 0.5.

According to some embodiments, the graduated score has an accuracy of above and a p-value for the accuracy of below 0.05 and an AUC of above 0.5. According to some embodiments, the graduated score has an accuracy of above 0.6 and a p-value for the accuracy of below 0.01 and an AUC of above 0.6.

According to some embodiments, the graduated score associated with sertraline has an accuracy of above 0.6 and a p-value for the accuracy of below 0.05.

In some embodiments, determining treatment response comprises identifying the subject as being resistant or susceptible to the treatment. In some embodiments, the antidepressant treatment response comprises resistance to an antidepressant treatment.

According to some embodiments, the antidepressant treatment comprises one or more medication selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, sertraline, escitalopram and vortioxetine and any combination thereof.

According to some embodiments, the antidepressant treatment comprises one or more medication selected from the group consisting of: citalopram, bupropion, sertraline, venlafaxine and escitalopram.

The present invention provides for the first time a strong predictive platform to help physicians in deciding whether to prescribe a specific antidepressant to a subject, or not.

The aforementioned surprising discoveries are a result of using advanced machine learning techniques in combination with expert knowledge in methods described herein.

According to some embodiments, the subject is diagnosed with depression. According to some embodiments, the subject in need of antidepressant treatment is diagnosed with depression.

According to some embodiments, the prediction of the responsiveness to antidepressant treatment is performed prior to initiation or at initiation of antidepressant treatment. In some embodiments, predicting the subject's responsiveness to antidepressant treatment comprises an accuracy of at least 58%. In some embodiments, predicting the subject's responsiveness to antidepressant treatment comprises an accuracy ranging between 58% and 70%. In some embodiments, predicting the subject's responsiveness to antidepressant treatment comprises an accuracy ranging between 50% and 70%. In some embodiments, predicting the subject's responsiveness to antidepressant treatment comprises an accuracy ranging between 55% and 85%. In some embodiments, predicting the subject's responsiveness to antidepressant treatment comprises an accuracy ranging between 58% and 98%.

The present invention further provides, in another aspect, a method for generating a predictor of antidepressant treatment, the method comprising selecting one or more features relevant to a subject's response to the antidepressant treatment based on expert knowledge, biological models and feature selection algorithms; ranking the selected features based on feature meta-ranking and/or one or more machine learning algorithms; and generating an ensemble predictor based on the feature selection and feature ranking. In some embodiments, the features comprise clinical features, demographics features, or any combination thereof.

The present invention further provides, in another aspect, generating a predictor of antidepressant treatment, by comprising selecting one or more features relevant to a subject's response to the antidepressant treatment based on expert knowledge, biological models and feature selection algorithms; ranking the selected features based on feature meta-ranking and/or one or more machine learning algorithms; generating an ensemble predictor based on the feature selection and/or feature ranking; and evaluating the ensemble predictor based on exponential modeling of the subject's treatment response, the exponential modeling based on an integrated analysis of changes in the subject's depression score and duration of treatment. In some embodiments, the features comprise clinical features, demographics features, or any combination thereof.

According to some embodiments, the initial ranking of selected one or more features is based on meta-analysis and is further revised based on the outcome of treatment versus predicted response.

According to some embodiments, the method further includes evaluating the ensemble predictor, based on exponential modeling of the subject's treatment response. According to some embodiments, the exponential modeling is based on an integrated analysis of changes in the subject's depression score and the duration of treatment.

According to some embodiments, the evaluation of ensemble predictor may (additionally or alternatively) be based on the subject's treatment response, wherein an improvement of at least 50% in the subject's depression score, after as compared to before treatment, is indicative of the subject being responsive to the treatment.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a schematic illustration of steps of an exemplary method for predicting antidepressant treatment response for a subject.

### DETAILED DESCRIPTION OF THE INVENTION

In the following description, various aspects of the disclosure will be described. For the purpose of explanation, specific configurations and details are set forth in order to provide a thorough understanding of the disclosure. However, it will also be apparent to one skilled in the art that the embodiments may be practiced without specific details being presented herein. Furthermore, well-known features may be omitted or simplified in order not to obscure the invention.

As used herein, the term “anti-depressant treatment” refers to drugs used in the treatment of patients suffering from depression. Antidepressants are known to influence the functioning of certain monoamine neurotransmitters, primarily serotonin, norepinephrine, and dopamine. Older medications, such as tricyclic anti-depressants (TCAs) and monoamine oxidase inhibitors (MAOIs), affect the activity of all these neurotransmitters simultaneously. Newer medications comprise selective serotonin reuptake inhibitors (SSRIs), norepinephrine-selective reuptake inhibitors (NRIs), Norepinephrine-Dopamine Reuptake Inhibitors (NDRIs), Serotonin-Norepinephrine-Dopamine Reuptake Inhibitor (SNDRI), Mirtazapine, Nefazodone, Trazodone, and Vortioxetine.

As used herein, the term “psychiatric disorders” refers to any psychiatric disorders including, but not limited to, depression, attention deficit disorder, schizophrenia, bipolar disorder, anxiety disorders, alcoholism, eating disorders such as anorexia and bulimia, phobias, dissociative disorders, insomnia, and borderline personality disorder.

As used herein, the terms “depression,” “depressive disorder,” and “mood disorder” interchangeably refer to a DSM-IV definition of depression. It is to be understood that depression comprises different subtypes such as Atypical depression (AD), Melancholic depression, Psychotic major depression (PMD), Catatonic depression, Postpartum depression (PPD), Seasonal affective disorder (SAD), Dysthymia, Depressive Disorder Not Otherwise Specified (DD-NOS), Recurrent brief depression (RBD), Major depressive disorder and Minor depressive disorder; which all fall under the scope of the invention.

Atypical depression (AD) is characterized by mood reactivity (paradoxical anhedonia) and positivity, significant weight gain or increased appetite (“comfort eating”), excessive sleep or somnolence (hypersomnia), a sensation of heaviness in limbs known as leaden paralysis, and significant social impairment as a consequence of hypersensitivity to perceived interpersonal rejection.

Melancholic depression is characterized by a loss of pleasure (anhedonia) in most or all activities, a failure of reactivity to pleasurable stimuli, a quality of depressed mood more pronounced than that of grief or loss, a worsening of symptoms in the morning hours, early-morning waking, psychomotor retardation, excessive weight loss, or excessive guilt.

Psychotic major depression (PMD), or simply psychotic depression, is the term for a major depressive episode, in particular of melancholic nature, wherein the patient experiences psychotic symptoms such as delusions or, less commonly, hallucinations.

Catatonic depression is a rare and severe form of major depression involving disturbances of motor behavior and other symptoms. Here, the person is mute and almost stuporous, and either is immobile or exhibits purposeless or even bizarre movements.

Postpartum depression (PPD) refers to the intense, sustained and sometimes disabling depression experienced by women after giving birth.

Seasonal affective disorder (SAD), also known as “winter depression” or “winter blues”, refers to depressive episodes coming on in the autumn or winter, and resolving in spring.

Dysthymia is a chronic, different mood disturbance where a person reports a low mood almost daily over a span of at least two years. The symptoms are not as severe as those for major depression.

Depressive Disorder Not Otherwise Specified (DD-NOS) refers to disorders that are impairing but do not fit any of the officially specified diagnoses.

Recurrent brief depression (RBD) is distinguished from major depressive disorder primarily by differences in duration. People with RBD have depressive episodes about once per month, with individual episodes lasting less than two weeks and typically less than 2-3 days.

As used herein the term “clinical feature” may refer to any non-genetic parameter influencing the subject's response to an antidepressant treatment. According to some embodiments, the term “clinical feature” may include physiological features (e.g., pain) and psychological features (e.g., anxiety), as further described herein below.

As used herein the term “demographic feature” may refer to any non-genetic parameter associated with an environment of the subject. According to some embodiments, the term “demographic feature” may include sociological features (e.g., marital status) and economical features (e.g., salary), as well as behavioral characteristics of the subject, as further described herein below.

As used herein the term “behavioral characteristics” may refer to any habit, routine, or custom of the subject. According to some embodiments, the term “behavioral characteristics” may include cell phone usage, internet usage habits, and one or more analyses derived from data extracted from cellphone and/or internet usage habits.

It is to be understood that “responsive” to antidepressant treatment as used herein does not necessarily mean that the subject will benefit from the antidepressant treatment, but rather that the subject is, in a statistical sense, more likely to belong to the class of patients that will benefit from the antidepressant treatment.

The term “classification algorithm” as used herein refers to methods that implement a model (classifier) for predicting a discrete category or class membership (target label), to which the data belong.

The term “non-linear classification algorithm” as used herein refers to non-linear models (classifiers) for prediction of class membership (target label).

The term “classification tree” as used herein refers to a non-linear model (classifier) for predicting class membership (target label) by constructing a decision tree, which repeatedly partitions the data, until it reaches a prediction of a discrete class (target labels).

The term “regression tree” as used herein refers to a non-linear method for predicting numerical values (target values). It involves constructing a decision tree for repeatedly partitioning the data, and predicting real-number target values.

The term “graduated score” as used herein refers to the total score that each subject receives from the method, which quantifies the predicted outcome. An accuracy above 0.5, represents a higher than the random chance of obtaining correct predictions. A p-value for the accuracy below 0.05, refers to a threshold used to limit the likelihood for false negative predictions to no more than 5% of the total number of predictions. The area under the curve (AUC) is used to determine which of the used models best classifies the data (target labels). An AUC of 0.5 is equal to a random prediction, whereas an AUC above 0.5, represent predictions where the true positive rates are greater than those that would be obtained by chance, and the false positive rates are minimized.

The term “random forest classifier” as used herein refers to a non-linear method for predicting a class membership (target label) by constructing multiple decision trees, and predicting the target labels based on the majority vote of the decision trees.

The term “boosting framework” as used herein refers to a method that combines multiple weak prediction models, which are sequentially added and weighted to produce a strong prediction model. This overall stronger model is used to predict the target values, in the case of regression, or target labels in the case of classification.

The term “sexual side effects” as used herein refers to side effects that can be caused by medication, which cause sexual dysfunction, decreased sexual desire, decreased sexual response and/or sexual ability.

As used herein, the term “efficacy” with regards to a subject's response to an antidepressant treatment refers to an improvement of 50% or more in the subject's depression score. Additionally or alternatively, the efficacy may be determined according to a depression curve taking into consideration both the depression score as well as time of treatment. The efficacy of the anti-depressant treatment is determined quantitatively by one or more rating scales, such as the Hamilton Rating Scale for Depression (HAM-D), QUICK INVENTORY OF DEPRESSIVE SYMPTOMATOLOGY (QIDS), Patient Health Questionnaire-9 (PHQ-9), Patient Health Questionnaire-8 (PHQ-8), Beck's Depression Inventory (BDI), Emotional State Questionnaire or Global Clinical Impression Scale. The HAM-D scale contains items that assess somatic symptoms, insomnia, working capacity and interest, mood, guilt, psychomotor retardation, agitation, anxiety, and insight. As used herein a 50% decrease in the HAM-D or the BDI score is considered an efficient treatment response. The degree of adverse side effects of anti-depressant treatment is determined quantitatively by the Udvalg Kliniske Undersogelser (UKU) Side Effect Rating Scale, the Frequency and Intensity of Side Effects Rating (FISER) or the Global Rating of Side Effects Burden (GRSEB) scales. Each possibility is a separate embodiment of the invention.

As used herein, the term “treatment resistant” or “resistant to antidepressant treatment” refers to a subject being unresponsive to at least two different antidepressant medications, such as but not limited to SSRIs such as but not limited to: citalopram, paroxetine, sertraline, zimelidine, escitalopram, indalpine, dapoxetine, fluvoxamine and fluoxetine; NRIs such as but not limited to: talopram, talsupram, reboxetine, viloxazine and atomoxetine; NDIRs such as but not limited to bupropion and desoxypipradrol; SNRIs such as but not limited to: edivoxetine, amedalin, desvenlafaxine, milnacipram, daledalin, venlafaxine, duloxetine, tandamine, lortalamine and levomilnacipran; piperidines such as but not limited to difemetorex and dexmethylphenidate; tetracyclic antidepressants such as but not limited to maprotiline and atypical antidepressants, such as, but not limited to Mirtazapine, Nefazodone, Trazodone, and Vortioxetine.

The term “expert knowledge” as used herein refers to knowledge acquired by continuous experience and through professional literature.

The term “biological model” as used herein refers to models based on biologically derived data.

The term “feature selection algorithm” as used herein refers to a method for identifying relevant and fewer predictors (features) with which to perform classification or regression predictions. According to some embodiments, either one of the “feature selection” and “feature extraction”, or both, may be used for feature reduction.

The term “feature meta-ranking” as used herein refers to ranking the features based on their importance, and overall effect on the prediction of the model.

The term “machine learning algorithm” as used herein refers to a construction of a method (algorithm) that can learn from and make predictions on data.

The term “ensemble predictor” as used herein refers to combining two or more prediction models, in order to improve the prediction model.

The term “exponential modeling” as used herein refers to a model that fits the data exponentially, this will suit cases where the data change by a fixed (or close to fixed) percentage.

The term “meta-analysis” as used herein refers to method for combining data from multiple studies or models. In some embodiments, the antidepressant treatment comprises at least one of the antidepressant medications selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, sertraline, and vortioxetine.

Venlafaxine (brand names: EFFEXOR, EFFEXOR XR, LANVEXIN, VIEPAX and TREVILOR) is an antidepressant of the serotonin-norepinephrine reuptake inhibitor (SNRI) class. This means it increases the concentrations of the neurotransmitters serotonin and norepinephrine in the synaptic cleft or synaptic gap.

According to some embodiments, the subject in need of the psychiatric drug may suffer from a psychiatric disorder selected from the group consisting of depression, attention deficit disorder, schizophrenia, bipolar disorder, anxiety disorders, alcoholism, eating disorders such as anorexia and bulimia, phobias, dissociative disorders, insomnia, and borderline personality disorder or any combination thereof. According to some embodiments, the subject is suffering from depression and the psychiatric drug is an anti-depressant. According to some embodiments, the antidepressant is selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, escitalopram, and vortioxetine and any combination thereof. According to some embodiments, the anti-depressant is citalopram.

In some embodiments, there is provided a method for predicting antidepressant treatment response for a subject in need thereof.

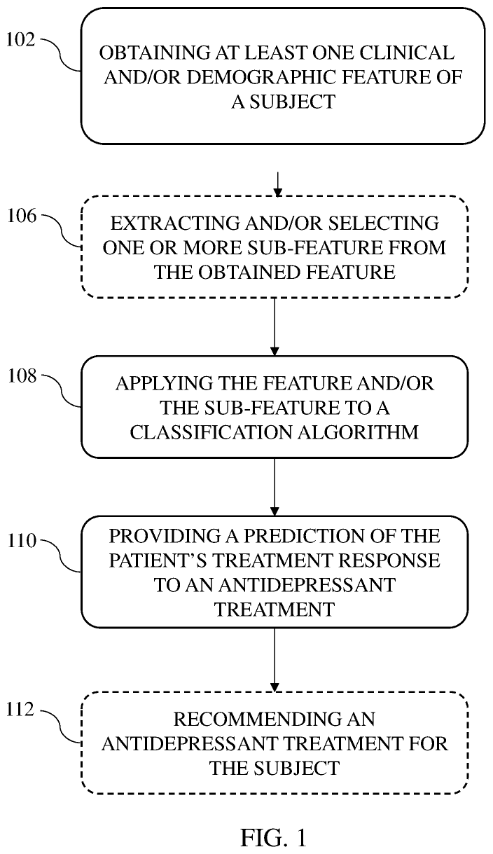

Reference is made to FIG. 1, which is a schematic illustration of steps of an exemplary method for predicting antidepressant treatment response for a subject.

In some embodiments, at step 102, the method comprises obtaining at least one clinical feature and/or at least one demographic feature of the subject. In some embodiments, the method may include applying the at least one clinical feature and/or at least one demographic feature to an algorithm configured to predict antidepressant treatment response for the subject. In some embodiments, the method comprises processing the at least one clinical feature and/or demographic feature. According to some embodiments, the algorithm may be configured to process the at least one clinical feature and/or demographic feature. Optionally, in some embodiments, at step 106, the method comprises extracting and/or selecting one or more sub-features from the obtained at least one feature. In some embodiments, the algorithm may be configured to extract and/or select one or more sub-features from the obtained at least one feature.

In some embodiments, at step 108, the method comprises applying the obtained feature and/or the sub-feature of the subject to a classification algorithm to. In some embodiments, the method may include applying the processed features and/or the extracted and/or selected sub-feature to the classification algorithm. In some embodiments, the classification algorithm is configured to provide a graduated score indicative of the treatment response to the antidepressant treatment. In some embodiments, the graduated score has an accuracy of above 0.5 and a p-value for the accuracy of below 0.05. In some embodiments, the graduated score has an AUC of above 0.5. In some embodiments, at step 110, the method comprises providing a prediction of the patient's treatment response to an antidepressant treatment. In some embodiments, the method comprises providing a prediction of the patient's treatment response based, at least in part, on the graduated score. Optionally, in some embodiments, at step 112, the method comprises recommending an antidepressant treatment for the subject. In some embodiments, the method comprises recommending an antidepressant treatment based, at least in part, on the on the graduated score and/or the at least one feature.

In some embodiments, the method comprises obtaining at least one clinical feature of a subject, as for example shown in step 102 of method 100. In some embodiments, the one or more clinical feature is selected from the group consisting of: severity level of problems in the upper gastro intestine, pains or aches at different body parts, presence and/or severity level of problems in the musculoskeletal/integument system, severity level of problems in the neurological system, fear of having an anxiety attack, reported feeling of unease, reported fear of having anxiety attack, history of psychotropic medications, poor treatment response to other antidepressants, reported troubling thoughts, fear of illness, and any combination thereof. In some embodiments, at least one demographic feature is selected from the group consisting of: employment status, residence, private health care insurance, age, marital status, one or more behavioral characteristics of the subject, and any combination thereof.

In some embodiments, the method comprises obtaining at least one demographic feature of a subject, as for example shown in step 102 of method 100. In some embodiments, the one or more demographic feature is selected from employment status, having private healthcare insurance, age, marital status, residence, one or more behavioral characteristics of the subject, and any combination thereof.

In some embodiments, the one or more behavioral characteristics are derived from data associated with computer usage and/or cell phone usage of the subject. In some embodiments, the one or more behavioral characteristics of the subject are monitored via a computer and/or cell phone of a subject. In some embodiments, the one or more behavioral characteristics of the subject are analyzed using data received from an electronic device used by the subject. For example, in some embodiments, the one or more behavioral characteristics are derived from data associated with an internet history of the subject. For example, in some embodiments, the one or more behavioral characteristics are derived from data associated with social media associated with the subject.

In some embodiments, the method comprises extracting and/or selecting one or more sub-feature from the obtained feature, as for example shown in step 106 of method 100. In some embodiments, any one of the clinical features and/or the demographic features can be divided into sub-features. In some embodiments, obtaining at least one of a clinical feature and/or a demographic feature of the subject comprises obtaining at least one sub-feature of a clinical feature and/or a demographic feature of the subject. In some embodiments, the method comprises obtaining a combination of sub-features associated with one or more of a clinical feature and/or a demographic feature. In some embodiments, the method comprises creating a new feature using a combination of sub-features associated with one or more of a clinical feature and/or a demographic feature.

In some embodiments, the sub-features comprise different levels of severity of a clinical feature of a subject. For example, the severity of one or more clinical features can be divided into a scale ranging from 0 to 10, thereby providing 11 sub-features associated with the severity of a clinical feature. In some embodiments, the sub-features comprise different locations associated with a clinical feature of a subject. For example, in some embodiments, a clinical feature of a subject is divided into a plurality of sub-features wherein each feature is associated with a different body part of the subject.

In some embodiments, a demographic feature of a subject is divided into sub-features associated with specific details of the demographic feature. In some embodiments, the sub-features comprise different statuses associated with demographic features of a subject. In some embodiments, a demographic feature of a subject is divided into sub-features associated with a range of complexity associate with a demographic feature. For example, a marital status can be divided into sub-features relating to number of children in custody of the subject.

In some embodiments, the sub-features are associated with a time and/or duration of the demographic feature. In some embodiments, the sub-features are associated with a history of a subject regarding the demographic feature. For example, in some embodiments, the demographic features such as employment status and/or having private healthcare insurance, are divided into sub-features associated with the time at which each of the demographic features had changed, and the status of the change itself.

In some embodiments, the method comprises obtaining a plurality of clinical and/or demographic features. In some embodiments, a plurality comprises at least two clinical and/or demographic features. In some embodiments, a plurality comprises at least three clinical and/or demographic features. In some embodiments, a plurality comprises at least five clinical and/or demographic features. In some embodiments, a plurality comprises at least ten clinical and/or demographic features.

In some embodiments, and as described in greater detail elsewhere herein, processing comprises applying one or more algorithms to the clinical and/or demographic features, as for example shown in step 108 of method 100. In some embodiments, the method comprises predicting a response for antidepressant treatment, as for example shown in step 110 of method 100.

In some embodiments, the method further includes predicting the risk of side effects resulting from treating the subject with the antidepressant treatment. In some embodiments, the predicted side effects are associated with one or more side effects corresponding to the Udvalg Kliniske Undersogelser (UKU) Side Effect Rating Scale, the Frequency and Intensity of Side Effects Rating (FISER) or the Global Rating of Side Effects Burden (GRSEB) scales. Each possibility is a separate embodiment of the invention. According to some embodiments, the side effects comprise one or more of sexual side effects, nausea, increased appetite, weight gain, fatigue, drowsiness, insomnia, dry mouth, blurred vision, and constipation, resulting from the treatment.

In some embodiments, the method comprises predicting treatment efficacy of an antidepressant drug in a subject in need thereof. In some embodiments, the method comprises obtaining at least one clinical and/or demographic feature of the subject. In some embodiments, the method comprises processing the at least one clinical and/or demographic feature by applying a classification algorithm. In some embodiments the classification algorithm is configured to provide a graduated score indicative of the treatment response to the psychiatric drug.

According to some embodiments, the method predicts the efficacy of the antidepressant treatment with at least 50% accuracy, at least 55% accuracy, at least 60 percent accuracy, at least 62% accuracy, at least 65% accuracy or at least 70% accuracy. Each possibility is a separate embodiment.

According to some embodiments, the method predicts the efficiency of the antidepressant treatment with at least 50% accuracy, at least 55% accuracy, at least 60 percent accuracy, at least 62% accuracy, at least 65% accuracy or at least 70% accuracy. Each possibility is a separate embodiment.

In some embodiments, the method comprises predicting a response for treatment using venlafaxine, in patients treated or intended to be treated with venlafaxine, by clinical and/or demographic features taken from the patients. According to some embodiments, the method predicts the efficiency of venlafaxine treatment with at least 65% accuracy or at least 70% accuracy. Each possibility is a separate embodiment. A person skilled in the art will find the present invention useful for deciding whether venlafaxine should be prescribed to a subject in need of antidepressant treatment.

In some embodiments, the method comprises characterizing a clinical condition related to a Central Nervous System (CNS) disease or disorder. In some embodiments, characterizing comprises selecting demographic and/or clinical features relevant to a subject affected by a CNS disease or disorder based on expert knowledge, biological models and feature selection algorithms. In some embodiments, characterizing comprises ranking the selected features based on feature meta-ranking and/or one or more machine learning algorithms. In some embodiments, characterizing comprises generating an ensemble predictor based on the feature selection and/or feature ranking. In some embodiments, characterizing comprises evaluating the ensemble predictor based on exponential modeling, the exponential modeling based on an integrated analysis of patients affected by a CNS disease or disorder.

In some embodiments, the method comprises predicting recommended antidepressant treatments for the subject. In some embodiments, the method comprises recommending an antidepressant treatment for the subject, as for example shown in step 112 of method 100. In some embodiments, the method comprises generating a predictor of response to antidepressant treatment. In some embodiments, generating a predictor of response comprises selecting clinical and/or demographic features relevant to a subject's response to the antidepressant treatment based on expert knowledge, biological models and/or feature selection algorithms. In some embodiments, generating a predictor of response comprises ranking the selected features based on feature meta-ranking and/or one or more machine learning algorithms. In some embodiments, generating a predictor of response comprises ranking the selected features using machine learning algorithms. In some embodiments, generating a predictor of response comprises generating an ensemble predictor based on the feature selection and/or feature ranking. In some embodiments, generating a predictor of response comprises evaluating the ensemble predictor based on exponential modeling of the subject's treatment response, the exponential modeling based on an integrated analysis of changes in the subject's depression score and duration of treatment.

Algorithm

In some embodiments, the method comprises applying at least one clinical feature, demographic feature, and/or sub-feature, to a classification algorithm, wherein the at least one sub-feature is extracted and/or selected from the at least one obtained clinical feature and/or demographic feature, as for example shown in step 108 of method 100. In some embodiments, the method comprises processing the at least one clinical feature and/or demographic feature by applying a classification algorithm thereto (or in other words, applying the at least one clinical feature and/or demographic feature to the classification algorithm). In some embodiments, the classification algorithm may be derived from a machine learning algorithm (and/or process). According to some embodiments, the classification algorithm may be a product of one or more machine learning algorithms.

In some embodiments, the method comprises selecting one or more machine learning models to apply the features and/or sub-features to, based on the available features and/or sub features. In some embodiments, the method comprises selecting one or more machine learning models to apply the features and/or sub-features thereto, based on a desired antidepressant treatments prediction, for example, for different and/or individual medications. In some embodiments, the machine learning algorithm is configured to utilize one or more specific features and/or sub-features associated with a specific antidepressant treatment.

In some embodiments, the method comprises applying a plurality of machine learning models for a single subject, thereby obtaining a plurality of results.

In some embodiments, the method comprises outputting a treatment recommendation for a subject, based on one or more result of applying features and/or sub-features associated with the subject to one or more machine learning model. In some embodiments, the method comprises outputting a treatment recommendation for a subject, based on one or more result outputted by the one or more machine learning model.

In some embodiments, processing the at least one clinical feature and/or the at least one demographic feature includes classifying thereof into at least two or more classes. In some embodiments, the at least two classes comprise: efficient and non-efficient. In some embodiments, the classification comprises a score indicative of the predicted degree of efficiency of the treatment.

In some embodiments, suitable classifiers include but are not limited to: Nearest Shrunken Centroids (NSC), Classification and Regression Trees(CART), ID3, C4.5, Multivariate Additive regression splines (MARS), Multiple additive regression trees(MART), Nearest Centroid (NC), Shrunken Centroid Regularized Linear Discriminate and Analysis (SCRLDA), Random Forest, Random Jungle, Boosting, Bagging Classifier, AdaBoost, RealAdaBoost, LPBoost, TotalBoost, BrownBoost, MadaBoost, XGBoost, LogitBoost, GentleBoost, RobustBoost, Support Vector Machine (SVM), partitioning around medoids (PAM), kernelized SVM, Linear classifier, Quadratic Discriminant Analysis (QDA) classifier, Naive Bayes Classifier and Generalized Likelihood Ratio Test (GLRT) classifier with plug-in parametric or non-parametric class conditional density estimation, k-nearest neighbor, Radial Base Function (RBF) classifier, Multilayer Perceptron classifier, Bayesian Network (BN) classifier, multi-class classifier adapted from binary classifier with one-vs-one majority voting, one-vs-rest, Error Correcting Output Codes, hierarchical multi-class classification, Committee of classifiers or other classifiers known and accepted in the art or any combination thereof. Each possibility is separate embodiment.

According to some embodiments, the classification algorithm comprises a non-linear classification algorithm. In some embodiments, the non-linear classification algorithm comprises an ensemble of classification and regression trees, more preferably wherein said ensemble of classification and regression trees, comprises a random forest classifier, support vector machine (SMV) or a boosting framework; or wherein the graduated score has an accuracy of above 0.5 and a p-value for the accuracy of below and an AUC of above 0.5.

In some embodiments, the method comprises applying at least one feature selection and/or feature extraction algorithm to the clinical and/or demographic features. In some embodiments, either one of the “feature selection” or “feature extraction” or both may be used for feature number reduction. It should be understood that the prediction model does not necessarily have to use the machine learning algorithms. In some embodiments, the method comprises obtaining sub-features from using feature selection and/or feature extraction algorithm. In some embodiments, the feature extracting and/or feature selection is configured to enable extracting and/or selecting of sub-features associated with a specific feature.

In some embodiments, the selection algorithm(s) may include one or more of the following techniques and algorithms: Feature similarity, Simulated Annealing, Ants Colony HillClimbing, iterated local search, PSO, Binary PSO or others.

Advantageously, the feature reduction, may facilitate shorter training times for the machine learning algorithms, simplification of the models, and provide modification ability by uses. According to some embodiments, the machine learning techniques/algorithms may include one or more of the following algorithms: Linear-Regression, KNN, K-Means, Random-Forest, SVM, Logistic-Regression, Decision-Tree, dimensionality reduction, Gradient boost and Adaboost, Naive-Bayes. In some embodiments, the method comprises preprocessing of the acquired signals by for example normalization, filtering, noise reduction, SNR optimization, domain transformations, statistical analysis, spectral analysis, wavelet analysis, or the like.

In some embodiments, the method comprises processing the at least one clinical and/or demographic feature. In some embodiments, processing the at least one clinical and/or demographic feature includes classification into at least two or more classes, for example efficient and non-efficient. According to some embodiments, suitable classifiers include but are not limited to: Nearest Shrunken Centroids (NSC), Classification and Regression Trees(CART), ID3, C4.5, Multivariate Additive regression splines (MARS), Multiple additive regression trees(MART), Nearest Centroid (NC), Shrunken Centroid Regularized Linear Discriminate and Analysis (SCRLDA), Random Forest, Random Jungle, Boosting, Bagging Classifier, AdaBoost, RealAdaBoost, LPBoost, TotalBoost, BrownBoost, MadaBoost, XGBoost, LogitBoost, GentleBoost, RobustBoost, Support Vector Machine (SVM), kernelized SVM, Linear classifier, Quadratic Discriminant Analysis (QDA) classifier, Naive Bayes Classifier and Generalized Likelihood Ratio Test (GLRT) classifier with plug-in parametric or non-parametric class conditional density estimation, k-nearest neighbor, partitioning around medoids (PAM), Radial Base Function (RBF) classifier, Multilayer Perceptron classifier, Bayesian Network (BN) classifier, multi-class classifier adapted from binary classifier with one-vs-one majority voting, one-vs-rest, Error Correcting Output Codes, hierarchical multi-class classification, Committee of classifiers or other classifiers known and accepted in the art or any combination thereof. Each possibility is separate embodiment. According to some embodiments, the non-linear classification algorithm is an ensemble of classification and regression trees. According to some embodiments, the non-linear classification algorithm is a random forest classifier or a boosting framework.

In some embodiments, the machine learning process includes a process of feature selection and dimensionality reduction wherein a great plurality of features, clinical features and/or demographic features undergo feature selection and dimensionality reduction to obtain a smaller amount of features relevant to providing an efficient prediction of the treatment response.

In some embodiments, the feature selection and dimensionality reduction techniques are selected from the group consisting of Multi Dimensional Scaling (MDS), Principal Component Analysis (PCA), Least Absolute Shrinkage and Selection Operator (LASSO), Sparse PCA (SPCA), Fisher Linear Discriminant Analysis (FLDA), minimum Redundancy Maximum Relevance (mRMR), Sparse FLDA (SFLDA), Kernel PCA (KPCA), ISOMAP, Locally Linear Embedding (LLE), Laplacian Eigenmaps, Diffusion Maps, Hessian Eigenmaps, Independent Component Analysis (ICA), Factor analysis (FA), Dimensionality Reduction (HDR), Sure Independence Screening (SIS), Fisher score ranks, t-test rank, Mann-Whitney U-test and any combination thereof, or as known and accepted in the art. Each possibility is separate embodiment. According to some embodiments, the feature selection technique applied during the machine learning process is Least Absolute Shrinkage and Selection Operator (LASSO).

In some embodiments, the method comprises selecting clinical and/or demographic features. In some embodiments, the method comprises selecting specific features by using expert knowledge, biological models and feature selection algorithms. In some embodiments, the method comprises ranking the features are ranked by feature meta-ranking. In some embodiments, feature meta ranking comprises ranking the features based on their importance, and overall effect on the prediction of the model, In some embodiments, feature meta ranking comprises ranking the sub-features based on their importance, and overall effect on the prediction of the model.

In some embodiments, the method comprises applying a machine learning algorithm to the features and/or to the meta-ranked features. In some embodiments, the method comprises applying a machine learning algorithm to the sub-features and/or to meta-ranked sub-features. In some embodiments, one or more machine learning algorithm corresponds to different features, In some embodiments, one or more machine learning algorithm corresponds to different sub-features. In sonic embodiments, the machine learning algorithm is selected based on the ranked features and/or sub-features.

In some embodiments, the method comprises applying the at least one clinical feature and/or demographic feature to a machine learning. In some embodiments, the method comprises applying one or more sub-features associates with the at least one clinical feature and/or demographic feature to a machine learning.

In some embodiments, applying a machine learning algorithm comprises applying an ensemble predictor on the at least one clinical feature and/or demographic feature In some embodiments, the ensemble predictor is derived from applying the machine learning algorithm on a data set of the at least one clinical feature and/or demographic feature obtained from patients with a known treatment response, thereby obtaining score indicative of the subject's treatment response. In some embodiments, the ensemble predictor and/or the machine learning algorithm is trained using a data set of the at least one clinical feature and/or demographic feature obtained from patients with a known treatment response. In some embodiments, the ensemble predictor and/or the machine learning algorithm is trained using labels for the data set, the labels indicating at least one known treatment response of patients from which at least one clinical feature and/or demographic feature were obtained. In some embodiments, applying machine learning algorithm comprises a step of feature selection. In some embodiments, the machine learning algorithm is configured to select one or more features and/or sub-features.

In some embodiments, the machine learning may be combined with expert knowledge. It is understood that this is a prerequisite for reliable feature selection since the number of possible features will always exceed the number of subjects included in the machine learning process. In some embodiments, 10-80 clinical features were included in the machine learning process. In some embodiments, 10-80 clinical features were included in the training process of the machine learning algorithm. In some embodiments, 10-80 demographic features were included in the machine learning process. In some embodiments, 10-80 demographic features were included in the training process of the machine learning algorithm. In some embodiments, the machine learning algorithm is trained to select features based on mathematical feature selection techniques. In some embodiments, the machine learning algorithm is trained to select features based on expert knowledge (e.g., for example, using a data set and labels). In some embodiments, the machine learning algorithm is trained to select features based on a combination of mathematical feature selection techniques and expert knowledge. Advantageously, feature selection based on a combination of mathematical feature selection techniques and expert knowledge enables reliable feature selection.

In some embodiments, the method comprises selecting clinical features and/or demographic features relevant to a treatment response based on expert knowledge, biological models and/or feature selection algorithms. In some embodiments, the method comprises ranking the selected features. In some embodiments, the method comprises ranking the selected features based on feature meta-ranking and/or one or more machine learning algorithms. In some embodiments, the method comprises generating an ensemble predictor based on the feature selection and/or feature ranking.

In some embodiments, the method comprises evaluating the ensemble predictor based on exponential modeling. In some embodiments, the method comprises evaluating the ensemble predictor based on exponential modeling, the exponential modeling based on an integrated analysis of the treatment response.

In some embodiments, the initial ranking of selected demographic and/or clinical features is based, at least in part, on meta-analysis and is further revised based on the outcome of treatment versus predicted response.

In some embodiments, the machine learning model is trained on a training set (and/or data set). In some embodiments, the training set comprises clinical features and/or demographic features of a plurality of subjects. In some embodiments, the training set comprises a plurality of sub-features associated with at least one of clinical features and/or demographic features of a plurality of subjects. In some embodiments, the training set comprises medical history records of the subjects wherein the medical history records are associated with antidepressant treatments. In some embodiments, the training data comprises data retrieved from the Sequenced Treatment Alternatives to Relieve Depression (STAR*D) and/or the Pharmacogenomic Research Network Antidepressant Medication Pharmacogenomic Study (PGRN-AMPS). In some embodiments, the training data associated with escitalopram is retrieved from the PGRN-AMPS.

In some embodiments, the training set comprises stratified data. In some embodiments, the training set comprises a random set of data comprising 70% of the STAR*D and/or the PGRN-AMPS data per treatment. In some embodiments, the training set comprises a set of data comprising between 40% and 90% of the STAR*D and/or the PGRN-AMPS data. In some embodiments, the training set comprises data associated with 180 to 2200 subjects. In some embodiments, the training set comprises data associated with 180 to 2200 subjects depending on the treatment. In some embodiments, the training set associated with each treatment is associated with a different number of subjects. In some embodiments, the training set associated with each treatment is associated with essentially 50% subjects who have responded to the treatment. In some embodiments, the training set associated with each treatment is associated with essentially 50% subjects who have not responded to the treatment.

In some embodiments, the training set comprises at least one of clinical features and/or demographic features associated with a plurality of subject. In some embodiments, the training set comprises at least one sub-feature associated with a clinical feature and/or a demographic feature of a subject. In some embodiments, the training set comprises a plurality of sub-features associated with clinical features and/or demographic features of the subjects.

In some embodiments, the training set comprises a plurality of labels associated with a plurality of specific antidepressant treatments. In some embodiments, the training set comprises a plurality of labels associated with side effects and/or success rates of the subjects for each antidepressant treatment. In some embodiments, the training set comprises a plurality of labels associated with an efficiency of specific antidepressant treatments.

In some embodiments, the machine learning model is trained using 5-fold cross-validation (CV). In some embodiments, the machine learning model is trained using 10-fold cross-validation (CV). In some embodiments, the machine learning model is trained using a range of 4 to 11-fold cross-validation (CV). In some embodiments, the machine learning model is trained using 3 to 7 repetitions. In some embodiments, the machine learning model is trained using 5 repetitions. In some embodiments, the number of fold cross-validation and/or of repetitions is different for one or more specific treatment. In some embodiments, the number of fold cross-validation and/or of repetitions corresponds with the number of subjects associated with a specific treatment.

In some embodiments, applying the classification algorithm further includes proving a graded score relating the level of treatment efficacy.

In some embodiments, the method further includes displaying or otherwise communicating the classification results. In some embodiments, the classification results may be displayed in a plurality of formats including printout, visual display cues, acoustic cues or the like. Each possibility is separate embodiment.

The foregoing description of the specific embodiments will so fully reveal the general nature of the invention that others can, by applying current knowledge, readily modify and/or adapt it for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not limitation. The means, materials and steps for carrying out various disclosed functions may take a variety of alternative forms without departing from the invention.

### EXAMPLES

### Example 1.

The method, as essentially set forth above, was applied on clinical and demographic data. The inputted clinical and demographic data included presence and/or severity level of any one of problems in the upper gastro intestine, pains or aches at different body parts, neurological issues, reported fear of having anxiety attack, history of psychotropic medications, poor treatment response to other antidepressants, reported troubling thoughts, reported sleep disorder, reported traumatic thoughts and effects, reported fear of public, open, and/or overpopulated spaces (Agoraphobia), employment status, residence, private health care insurance, age, and marital status.

The inputted data was applied to a machine learning algorithm trained as described in greater detail above. The machine learning models (or in other words, the machine learning algorithms) used clinical and/or demographic features of subjects in order to predict the ideal treatment per subject for Citalopram, Bupropion, Venlafaxine, Sertraline and Escitalopram. The machine learning algorithm outputted the accuracy and the balanced accuracy of the model for Bupropion, Venlafaxine, Sertraline, Escitalopram and Citalopram, as presented in the charts below.

| | | | | | Escitalo- |
|---|---|---|---|---|---|
| | Bupropion | Venlafaxine | Sertraline | Citalopram | pram |
| Accuracy | 70.69% | 64.81% | 63.64% | 58.57% | 62.96% |
| Balanced | 70.69% | 64.48% | 63.56% | 58.57% | 63.13% |
| Accuracy | | | | | |

| | | Standard |
|---|---|---|
| | Average | Deviation |
| Accuracy | 64.13% | 4.36% |
| Balanced | 64.08% | 4.34% |
| Accuracy | | |

As seen in the chart above, the average balanced accuracy of the model was 64.08%, with a standard deviation of 4.34%.

1. A method for predicting antidepressant treatment response for a subject in need thereof, the method comprising:
obtaining at least one clinical feature and/or demographic features of the subject; and
processing the at least one clinical feature and/or demographic feature by applying the at least one clinical feature and/or demographic feature to a classification algorithm, the classification algorithm configured to provide a graduated score indicative of the treatment response to the antidepressant treatment; and
provide a prediction of the patient's treatment response based on the graduated score, wherein the prediction does not require taking into consideration genetic information of the subject.

2. The method of claim 1, wherein one or more clinical feature is selected from the group consisting of severity level of problems in the upper gastro intestine, reported pains or aches in different body parts, reported neurological issues, reported fear of having an anxiety attack, fear of public, open and/or over populated places, reported traumatic thoughts and effects, having a history of psychotropic medications, having a poor treatment response to other antidepressants, reported troubling thoughts, sleep disorders, and any combination thereof.

3. The method of claim 1, wherein the one or more demographic feature is selected from employment status, having private healthcare insurance, age, marital status, residence, and any combination thereof.

4. The method according to claim 1, wherein applying a classification algorithm comprises applying the at least one clinical feature and/or demographic feature to a machine learning, wherein applying a machine learning comprises applying an ensemble predictor on the at least one clinical feature and/or demographic feature, wherein the ensemble predictor is derived from applying the machine learning algorithm on a data set of the at least one clinical feature and/or demographic feature obtained from patients with a known treatment response, thereby obtaining score indicative of the subject's treatment response.

5. The method of claim 4, further comprising generating the ensemble by applying one or more clinical and/or demographic features of patients with a known treatment response to a machine learning algorithm.

6. The method of claim 4, further comprising adjusting the ensemble predictor based on an actual treatment response versus the predicted treatment response.

7. The method of claim 1, further comprising determining side effects of the antidepressant treatment.

8. The method of any claim 1, wherein the classification algorithm comprises a non-linear classification algorithm.

9. The method of claim 8, wherein the non-linear classification algorithm comprises an ensemble of classification and regression trees.

10. The method of claim 9, wherein said ensemble of classification and regression trees, comprises a random forest classifier or a boosting framework.

11. The method of claim 1, wherein the graduated score has an accuracy of above 0.5 and a p-value for the accuracy of below 0.05 and an AUC of above 0.5.

12. The method of claim 1, wherein the antidepressant treatment comprises at least one of the antidepressant medications selected from the group consisting of: citalopram, paroxetine, sertraline, zimelidine, escitalopram, indalpine, dapoxetine, fluvoxamine, fluoxetine, talopram, talsupram, reboxetine, viloxazine, atomoxetine, bupropion, desoxypipradrol, edivoxetine, amedalin, desvenlafaxine, milnacipram, daledalin, venlafaxine, duloxetine, tandamine, lortalamine, levomilnacipran, difemetorex, dexmethylphenidate, maprotiline, mirtazapine, nefazodone, trazodone, sertraline, and vortioxetine.

13. The method of claim 1, wherein said at least one clinical feature and/or demographic features of the subject comprises at least one sub-feature.

14. The method of claim 1, wherein said at least one clinical feature and/or demographic features of the subject comprises a plurality of sub-features associated therewith.

15. A method for predicting antidepressant treatment response for a subject in need thereof, the method consisting essentially of:
obtaining at least one clinical feature and/or demographic features of the subject; and
processing the at least one clinical feature and/or demographic feature by applying the at least one clinical feature and/or demographic feature to a classification algorithm, the classification algorithm configured to provide a graduated score indicative of the treatment response to the antidepressant treatment; and
provide a prediction of the patient's treatment response based on the graduated score.

16. A method for associating a subject with a specific treatment response, the method, comprising:
selecting clinical features and/or demographic features relevant to the treatment response based on expert knowledge, biological models and feature selection algorithms;
ranking the selected features based on feature meta-ranking and/or one or more machine learning algorithms;
generating an ensemble predictor based on the feature selection and/or feature ranking; and
evaluating the ensemble predictor based on exponential modeling, the exponential modeling based on an integrated analysis of the treatment response, wherein the evaluating does not require taking into consideration genetic information of the subject.

17. The method of claim 16, further comprising generating the ensemble by applying one or more clinical and/or demographic features of patients with a known treatment response to a machine learning algorithm.

18. The method of claim 16, further comprising adjusting the ensemble predictor based on an actual treatment response versus the predicted treatment response.
